(19) United States
(12) Patent Application Publication
(10) Pub. No.: US 2005/0256376 A1
(43) Pub. Date: Nov. 17, 2005

(54) Connector for use with multilumen tubing

(75) Inventors: Yakov Bar-Or, Haifa (IL); Amram Aizenfeld, Kibbutz Ramot Menashe (IL); Golan Salman, Tirat Hacarmel (IL); Omer Shazifi, Haifa (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Haifa (IL)

(21) Appl. No.: 11/121,694

(22) Filed: May 4, 2005

Related U.S. Application Data
(60) Provisional application No. 60/570,609, filed on May 13, 2004.

(51) Int. Cl. A61B 1/12

(52) U.S. Cl. 600/156; 600/153

(57) ABSTRACT

A connector is described, which is suitable for establishing flow communication between passages of a multilumen tubing used with an endoscopic apparatus and between tubes for supplying fluid medium to the multilumen tubing. The connector is adapted for insertion and retraction of a surgical instrument therethrough. The connector comprises a main body portion provided with a through-going bore, which longitudinally extends along the main body portion. The main body portion further comprises a plurality of lateral ports, which are in flow communication with the through going bore. The bore is adapted for insertion thereinto and removal therefrom of a proximal end of the multilumen tubing. The lateral ports are adapted for receiving thereinto the tubes for supplying the fluid medium.

### FIELD OF THE INVENTION

The present invention relates generally to the field of endoscopy and specifically to endoscopic apparatus used for endoscopic procedures during which a flexible tube is inserted into a body passage for examination of its interior for abnormalities. More particularly, the present invention refers to a connector for disposable multilumen tubing, which is used specifically, but not exclusively, with an endoscope, having a disposable sleeve covering the tube and upon inflation enabling advancement of the colonoscope within the colon.

### BACKGROUND OF THE INVENTION

There are known endoscopes employing inflatable flexible sleeves for propulsion endoscope within colon.

Voloshin (U.S. Pat. No. 6,485,409) discloses an endoscope, which comprises an endoscopic probe, a bending section for directing the probe within the colon (steering unit), an insertion tube and a flexible covering sleeve or a sheath, which is coupled proximally to the probe. The bending section of the endoscope is located behind the probe. The sleeve is attached to the endoscope in such a manner that its folded section is retained between a cap and an internal spindle, which are located between the insertion tube and the probe head. When inflated, the folded section unfolds over a flange of the internal spindle and an inner portion of the sleeve is pulled in a distal direction.

In PCT/IL03/00661 an endoscope is described which employs a flexible inflatable sleeve, which before inflation is retained within a dispenser. The dispenser employed in this endoscope has entry and exit ports defining a transit passage through which the endoscope may pass. The dispenser is adapted to capture the sleeve as the endoscope is retracted through the transit passage in a proximal direction. In another embodiment, the dispenser includes an external sleeve fixed to the dispenser and this external sleeve is adapted to be extended from the dispenser when the endoscope is retracted so that the external sleeve covers the flexible sleeve. By virtue of this provision any contamination on the flexible sleeve remains within the external sleeve and does not contact the endoscope or any other objects or areas outside the patient's body. After the endoscope has been removed entirely from the flexible sleeve, the dispenser together with the external sleeve is discarded.

It is mentioned in the above reference that the endoscope is provided with an internal sleeve, which is also known as a multilumen tubing, since it is usually fitted with appropriate passages or lumens as required for irrigation, suction and for passing endoscopic tools therethrough. For functioning the endoscope, the proximal end of the multilumen tubing is detachably connected through a dedicated connector to respective tubes or hoses, which supply water, air and vacuum from the control unit. Furthermore, the connection has also a dedicated port for insertion and evacuation therethrough of a surgical instrument, which extends along one of the passages of the multilumen tubing.

Unfortunately the known in the art connectors are of relatively complicated design, they comprise several components and require dedicated sealing means to seal hoses entering through ports of the fitting for supplying water, air or vacuum. Furthermore, in order to connect in an air-tight fashion the multilumen tubing with the fitting and at the same time to ensure that water, air or vacuum enters into respective channels of the multilumen tubing, one should locate the multilumen tubing in such a manner that its passages are brought in alignment with the corresponding hoses. This renders preparation of the endoscopic apparatus for endoscopic procedure complicated and inconvenient.

### SUMMARY OF THE INVENTION

The object of the present invention is to provide a new and improved disposable connector for use with the multilumen tubing of an endoscopic apparatus.

The further object of the invention is to provide a new, disposable connector, which has very simple construction, which is convenient in use and which does not require bringing the tubes originating from the control unit in alignment with the passages of the multilumen tubing.

For a better understanding of the present invention as well of its benefits and advantages, reference will now be made to the following description of its embodiments taken in combination with the accompanying drawings.

### BRIEF DESCRIPTION OF THE DRAWINGS

FIG. 1 depicts a general view of endoscopic apparatus of the invention, preferably a colonoscopic apparatus and its main components

FIG. 2 is an enlarged perspective view of the proximal end of the multilumen tubing used with the connector of the present invention

FIG. 3 is an enlarged perspective view of the connector of the present invention

FIG. 4 is a cross-sectional view of the connector according the present invention with connection tubes for supply water, air and vacuum.

FIG. 5 is a cross-sectional view of the connector depicted in FIG. 4 with surgical tool inserted thereinto.

### DETAILED DESCRIPTION OF THE INVENTION

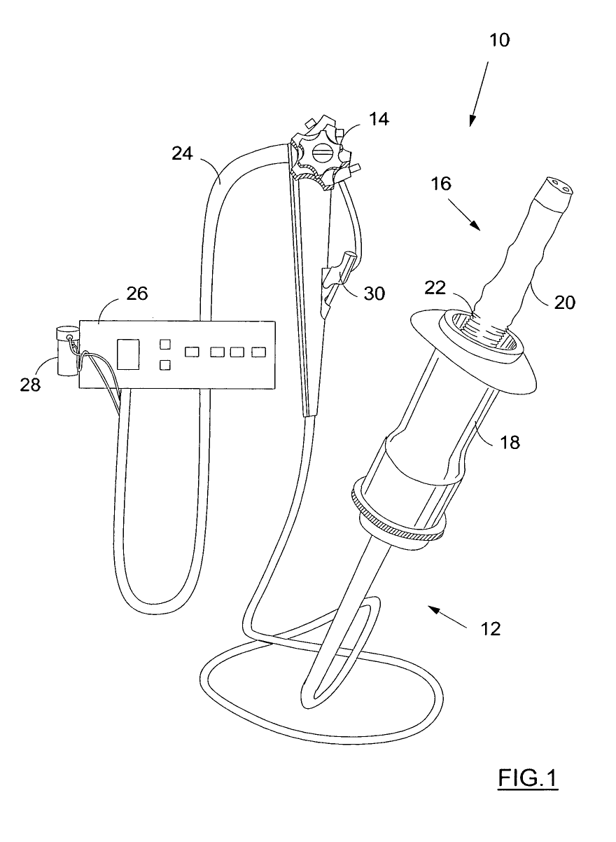

With reference to FIG. 1 the endoscopic apparatus of the present invention is shown preferably as a colonoscopic apparatus 10 with its following main components. The apparatus comprises an endoscope having insertion tube 12 with its proximal section connected to an operation handle 14 and with its distal section 16 inserted in and protruding from a disposable dispenser 18.

It is seen also in FIG. 1 that a sleeve covers distal section 16 of the endoscope. That part of the sleeve, which is seen in FIG. 1, comprises a frontal non-inflatable portion 20 and a rear, folded portion 22. The frontal portion of the sleeve covers the distal section of the endoscope and its head. The frontal portion does not inflate when the endoscope advances within the colon. The rear portion covers the insertion tube and unfolds when air or another fluid medium is supplied to the sleeve. By virtue of this provision the feeding out sleeve propels the endoscope within the body passage. Explanation of this phenomenon can be found in the above referred-to references. The endoscope of the present invention is of similar type in the sense that it employs the same propelling mechanism, which is based on inflation of the flexible disposable sleeve coupled to the endoscope. It should be appreciated, however, that the endoscope of the present invention is not limited merely to colonoscopy. It can be employed in any other medical procedure requiring insertion of a probe in a body passage for inspection of its interior.

It is seen also in FIG. 1, that the handle is connected by appropriate umbilical duct 24 to a control unit 26 provided with a source of compressed air for inflating and venting the sleeve. Proximate to the control unit a flask 28 is provided, which is filled with water, to be supplied under pressure into the colon for irrigation. Water and air are supplied to the handle through dedicated tubes attachable to the umbilical duct.

One should bear also in mind that within the insertion tube are provided various devices, which are necessary for proper functioning of the endoscope. These devices are known per se. Among such devices one can mention vertebrae and strings, which can be manipulated by the handle, and a multilumen tubing with appropriate passages for supplying water, as required for irrigation, or vacuum, as required for suction. The multilumen tubing also is provided with a dedicated passage for introducing surgical instruments into the colon as might be required during the endoscopic procedure. Along the insertion tube might extend also a dedicated tube through which air is supplied as required for inflating the sleeve.

The multilumen tubing extends through the endoscope and through the handle to a connector 30, which provides flow communication between lumens of the multilumen tubing and tubes passing through the umbilical duct tubes and supplying air and vacuum from the control unit and water from the flask.

The multilumen tubing and connector are manufactured from plastic material. It would be advantageous if they are cheap, disposable items, which are discarded at the end of the endoscopic procedure after the endoscope has been evacuated form the body passage. By virtue of this provision preparation for the new endoscopic procedure is simple, convenient and fast and it is not associated with spreading of any contamination picked up from the body passage during the endoscopic procedure.

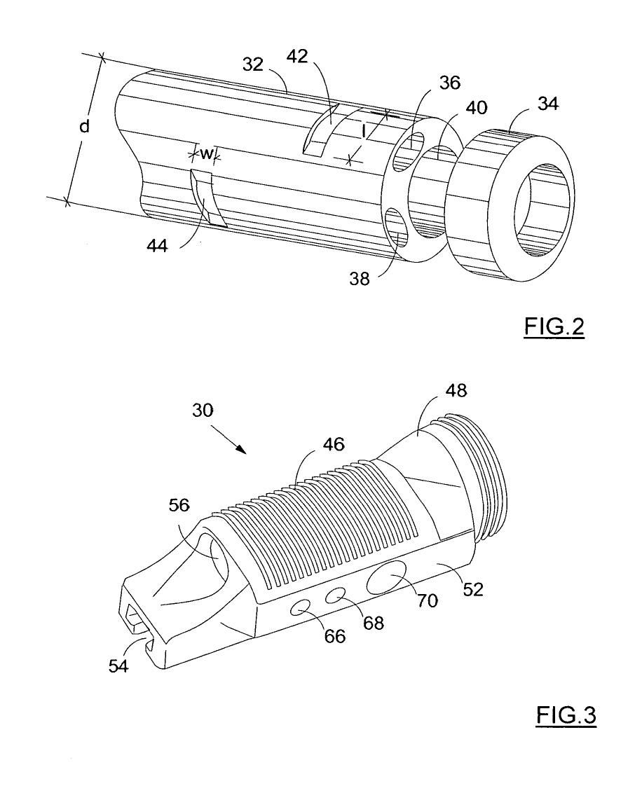

Referring now to FIG. 2 it is shown a proximal portion 32 of the multilumen tubing employed with the connector of the invention. The proximal portion terminates by a proximal end 34. The multilumen tubing constitutes an elongated tubular member, having single integral body through which extend longitudinally axial working passages 36, 38 and 40. The working passages 36 and 38 are of a similar shape and size and have preferably an elliptical cross-section. The passages 36,38 extend almost along the entire length of the multilumen tubing and are sealed at the most proximal end 34. One of these passages is intended for supplying air and the other one for supplying of water. The third axial passage 40 is of a larger cross-section and has a circular cross-sectional configuration. This passage is intended for supplying vacuum or for insertion of a surgical tool therethrough. The third passage extends along the entire length of the multilumen tubing and its open end is seen in FIG. 2. Location of the passages within the multilumen tubing as well as their cross-sectional configuration is selected in such a manner that an outside diameter d of the multilumen tubing is kept at a minimum, which ensures that the wall thickness would be still sufficient to resist collapsing due to the vacuum supplied through the passage 40.

On the outside periphery of the multilumen tubing and in vicinity to the proximal end 34 two tangentially directed cuts 42,44 are made, which are defined by a width dimension w and by a length dimension 1. In practice these dimensions are selected to allow communication with respective axial passages 36 and 38. The tangentially directed cuts provide, in fact, radial ports, through which a fluid medium can enter in the respective axial passages of the multilumen tubing. In practice the multilumen tubing is manufactured from an elastomeric material, e.g. PVC of medical grade. The outside diameter d of the multilumen tubing is about 6.2 mm, the minimal wall thickness between the outside periphery and the passage is about 0.2-0.3 mm, the width dimension w is about 1.5-1.6 mm and the length dimension l is about 3 mm.

Referring now to FIGS. 3,4 construction of the connector will be explained. Connector 30 of the invention is made of an elastometic material, which should have resiliency of about 60 Shor. In practice this material can be silicon rubber or any other elastomeric plastic material.

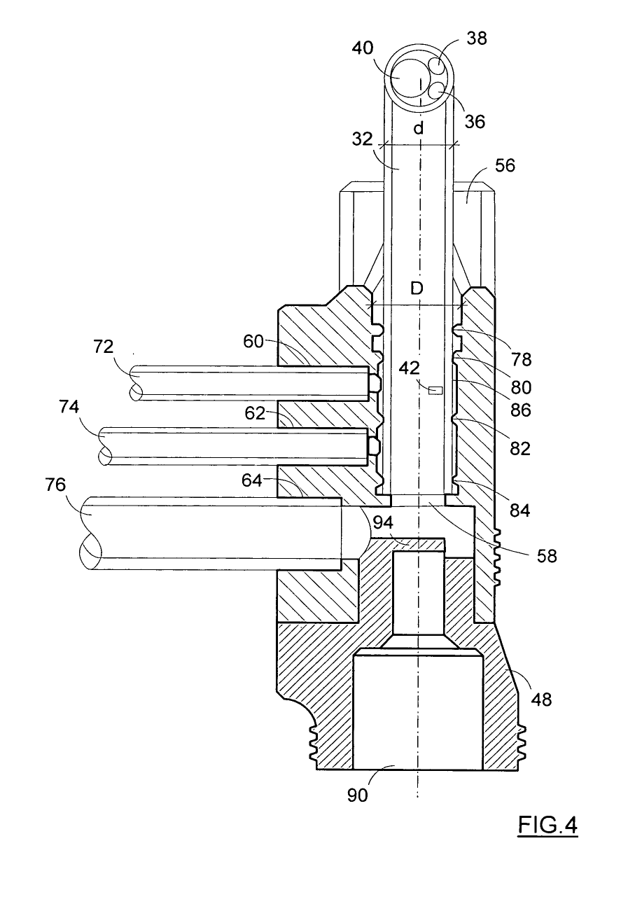

The connector comprises a main body portion 46 and attached thereto a proximal port 48. The main body portion comprises a lower portion 50, which is integral with an upper portion 52. The lower portion has an U-shaped configuration defining a longitudinal groove 54 adapted for sliding along an appropriate protrusion provided on a lateral port of the handle 14. By virtue of this provision the connector can be detachably connected to the handle as seen in FIG. 1. Within the upper portion of the housing a longitudinally extending through-going bore is provided. It is seen in FIG. 4, that the bore has an inside diameter D and it extends between an exit opening 56 and an entry opening 58. This bore is intended for insertion and removal of the proximal end of the multilumen tubing 32 and for insertion and removal of a surgical instrument. Directed transversally with respect to the bore and communicating therewith three lateral ports 60,62,64 are provided, which respective openings 66,68,70 are seen in FIGS. 3,4.

Two of these ports (60 and 62) are intended for insertion thereinto of tubes 72,74, which respectively supply water and air to passages 36,38 of the multilumen tubing. The third port 64 is intended for insertion thereinto of a tube 76 through which vacuum is supplied to passage 40 of the multilumen tubing. In practice the ports 60,62 are of similar diameter, which is less than the diameter of the port 64.

The inside diameter D of the through going bore is selected in such a manner, that it is more than the outside diameter d of the multilumen tubing bore so as to allow insertion of the proximal end 34 of the multilumen tubing into the connector. In practice the inside diameter D is 6.2-6.3 mm.

Formed on the circumference of the through going bore a plurality of discrete annular protrusions 78,80,82,84 are provided. All those protrusions reduce the inside diameter D from 6.2-6.3 mm to about 5.5 mm, which is less than the outside diameter d of the multilumen tubing. By virtue of this provision and due to resiliency of the elastomeric material, the annular protrusions resiliently deform and reliably seal the multilumen tubing when it is being inserted in the connector. By virtue of this provision airtight connection between the multilumen tubing and the connector is provided without necessity to use separate sealing rings or any other dedicated sealing components. This renders construction of the connector very simple. The protrusions are intended also to provide two annular pockets, communicating with lateral ports for supply water and air. It is seen in FIG. 4, that an annular pocket 86 is formed between the annular protrusions 80,82 and an annular pocket 88 is formed between annular protrusions 82,84. It also seen that lateral port 60 is in flow communication with the pocket 86 and lateral port 62 is in flow communication with the pocket 88. The distance between the adjacent protrusions is selected in such a manner that the lateral ports open immediately in the pockets and supply fluid medium in thereinto.

It can be readily appreciated that when the proximal end of the multilumen tubing is inserted in the connector as seen in FIG. 4, the tangential cuts are exposed to the fluid medium located in the annular pockets and this fluid medium can enter through the tangential cuts in the respective axial passages 36,38. It can be also appreciated that the fluid medium fills the entire annular space of the corresponding pocket and thus entrance of the fluid medium in the axial passage would be always ensured irrespective of an angular orientation of the multilumen tubing with respect to the bore's circumference.

Vacuum is supplied to the open end of the passage 40 of the multilumen tubing through entry opening 58.

By virtue of the connector of the present invention supply of all necessary fluid media to the multilumen tubing is provided, while preparation of the multilumen tubing to the endoscopic procedure becomes very simple and convenient since it requires now merely the insertion of the proximal end of the multilumen tubing in the exit opening of the connector.

In practice the rear part of the connector, i.e. its proximal port 48, is manufactured from the same elastomeric material as the main body portion. The proximal port is connected to the main body portion by any suitable means, e.g. gluing. The situation, when the proximal port is a separate item connectable to the main body portion is only an illustrative example. One can contemplate a situation when the proximal port and the main body portion constitute an integral item.

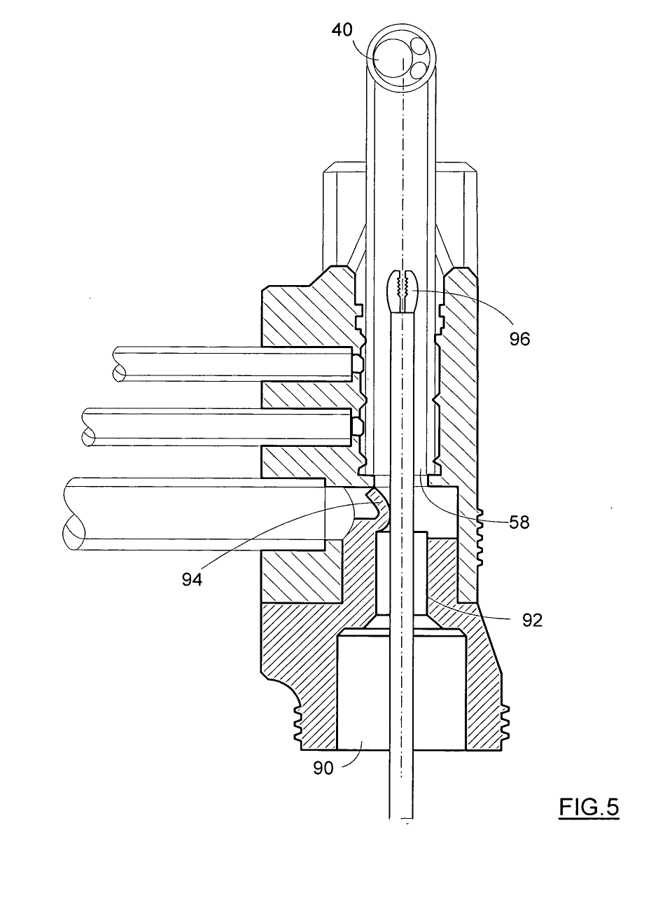

The proximal port is provided with an entrance opening 90 for insertion therethrough and for evacuation therefrom of a surgical instrument as will be explained further with reference to FIG. 5. The entrance terminates by a narrowing section 92. A thin, flexible partition 94 is provided, which is resiliently collapsible so as to open or close the narrowing passage and thus to prevent or permit flow communication between the entry opening of the bore and the entrance opening of the proximal port. In the situation shown in FIG. 4, the multilumen tubing is inserted within the connector, however the surgical instrument is not inserted within the connector. Resilient partition 94 is configured and dimensioned so as to reliably close the entrance port and to prevent flow communication with the through-going bore, while supply of a fluid medium or vacuum to respective passages of the multilumen tubing is possible.

In FIG. 5 is depicted a situation where a surgical instrument, e.g. biopsy forceps 96, is inserted through the entrance opening 90 within the connector and has been already advanced through the entry opening 58 so as to enter passage 40 of the multilumen tubing. The instrument is to be advanced further through the passage 40 in order to take a tissue example within the patient's body during the endoscopic procedure. It can be appreciated that in this situation no vacuum is supplied to the passage 40. During the initial step of advancing the instrument through the entrance opening the forward end of the instrument reaches resilient partition 94, pushes it forward and urges it to collapse as seen in FIG. 5. Now the instrument can be advanced further along the passage 40 of the multilumen tubing. At the end of the endoscopic procedure when the instrument is retracted from the connector, the partition returns to its closed position due to the resiliency of the elastomeric material and the proximal port is closed again.

It should be appreciated that the above-disclosed connector can be employed not only in connection with an endoscope, which is propelled by an inflatable sleeve. It can be used with any other endoscopes, in which it is required to supply fluid medium to passages of multilumen tubing or to advance surgical instrument therethrough.

It should be also appreciated that the invention is not limited to the above-described embodiments and that changes and one ordinarily skilled in the art can make modifications without deviation from the scope of the invention, as will be defined in the appended claims.

For example instead of a resilient partition, which is bendable as shown in FIG. 5, one could use a thin resilient wall, which is pierceable by the surgical instrument when it is advanced towards the entry opening.

When used in the following claims, the meaning of terms “comprise”, “include”, “have” and their conjugates is “including but not limited to”.

It should also be appreciated that the features disclosed in the foregoing description, and/or in the following claims, and/or in the accompanying drawings may, both separately and in any combination thereof, be material for realizing the present invention in diverse forms thereof.

1. A connector for establishing flow communication between passages of a multilumen tubing used with an endoscopic apparatus and between tubes for supplying fluid medium to the multilumen tubing during an endoscopic procedure, said connector being also adapted for insertion and retraction of a surgical instrument therethrough.

2. The connector as defined in claim 1, comprising a main body portion provided with a through-going bore longitudinally extending therealong between an entry opening and an exit opening, said main body portion further comprising a plurality of lateral ports, which are in flow communication with the said bore, wherein the bore permits insertion thereinto and removal therefrom of a proximal end of the multilumen tubing and the lateral ports receive thereinto said tubes for supplying the fluid medium.

3. The connector as defined in claim 2, wherein the main body portion being provided with a proximal port for insertion and retraction of the surgical tool, said proximal port being in flow communication with the entry opening of the through-going bore.

4. The connector as defined in claim 3, wherein said main body portion is made of a resilient plastic material.

5. The connector as defined in claim 4, wherein said bore has an inside diameter greater than an outside diameter of the multilumen tubing, wherein a portion of the through-going bore being provided with a plurality of discrete annular protrusions for sealing the multilumen tubing when it is inserted in the through-going bore.

6. The connector as defined in claim 2, wherein said main body portion being detachably connectable to an operating handle of the endoscopic apparatus.

7. The connector as defined in claim 5, wherein said proximal port being provided with an entrance opening and with a passage for communicating with the entry opening of the through-going bore, wherein said passage including a partition to seal the entry opening of the through-going tube from the proximal port when no surgical instrument is inserted in the proximal port.

8. The connector as defined in claim 2, wherein said lateral ports supply the fluid medium tangentially with respect to the multilumen tubing.

9. The connector as defined in claim 8, wherein said lateral ports supply water, air and vacuum.

10. The connector as defined in claim 2, wherein between adjacent annular protrusions are formed annular pockets, which are in flow communication with the radial ports.

11. The connector as defined in claim 1, wherein said connector is disposable.

12. A multilumen tubing comprising axial passages for supplying fluid medium to an endoscopic apparatus and an axial passage for insertion and retraction of a surgical instrument, said multilumen tubing further comprising a single, integral body having a proximal end fitted with radial openings made on its circumference, said openings being in flow communication with the passages for supplying the fluid medium.

13. The niultilumen tubing as defined in claim 12, wherein said radial openings comprise tangentially directed rectangular cuts.

14. The multilumen tubing as defined in claim 12, wherein in said multilumen tubing the passage for insertion and retraction of the surgical instrument terminates by an axially open end.

15. The multilumen tubing as defined in claim 12, wherein said passages for supplying the fluid medium have an elliptical cross section and said passage for insertion and retraction of the surgical instrument has a circular cross section.

16. The multilumen tubing as defined in claim 12, wherein said multilumen tubing being made from an elastomeric material.

17. The multilumen tubing as defined in claim 12, wherein said multilumen tubing being disposable.

18. An endoscopic apparatus comprising
an endoscope,
a multilumen tubing insertable into said endoscope, said multilumen tubing being provided with passages for supplying fluid medium to the endoscope and with a passage for insertion and retraction of a surgical instrument,
tubes for supplying fluid medium to the passages of the multilumen tubing,
a connector establishing flow communication between passages of the multilumen tubing and said tubes for supplying fluid medium, said connector enabling said surgical instrument being inserted into and retracted from said multilumen tubing.

19. The endoscopic apparatus as defined in claim 18, in which said connector comprises a main body portion provided with a through-going bore longitudinally extending therealong between an entry opening and an exit opening, said main body portion further comprising a plurality of lateral ports, which are in flow communication with said bore, wherein the bore permits insertion thereinto and removal therefrom of a proximal end of the multilumen tubing and the lateral ports receive thereinto said tubes for supplying the fluid medium.

20. The endoscopic apparatus as defined in claim 19, in which the main body portion being provided with a proximal port for insertion and retraction of the surgical tool, said proximal port being in flow communication with the entry opening of the through-going bore.
